(19) United States
(12) Patent Application Publication
(10) Pub. No.: US 2026/0027080 A1
(43) Pub. Date: Jan. 29, 2026

(54) CATECHIN CONTAINING FORMULATIONS FOR USE IN TREATING DIABETIC CARDIOMYOPATHY

(71) Applicant: ADAMAS BIOTECH S.R.L ., Maglie Lecce (IT)

(72) Inventor: Saverio BETTUZZI, Maglie Lecce (IT)

(21) Appl. No.: 18/998,761

(22) PCT Filed: Jul. 28, 2023
(86) PCT No.: PCT/EP2023/071023

§ 371 Date: Jan. 27, 2025

(30) Foreign Application Priority Data
Jul. 28, 2022 (GB) 2211063.9

(51) Int. Cl. A61K 31/353; A61P 9/00

(52) U.S. Cl. CPC A61K 31/353; A61P 9/00

(57) ABSTRACT

There is provided inter alia a pharmaceutical formulation comprising a catechin component for use in the treatment or prevention of diabetic cardiomyopathy.

The present invention relates to a formulation for use inter alia in the treatment or prevention of diabetic cardiomyopathy. In particular, the invention relates to a pharmaceutical formulation comprising a catechin component for use in the treatment of diabetic cardiomyopathy.

### BACKGROUND

Cardiomyopathy is a group of diseases that affect the heart muscle. Cardiomyopathies are frequently associated with an absence of symptoms during early stages however disease progression is associated with shortness of breath, fatigue, irregular heart beat and fainting, ultimately culminating in a severe risk of heart failure and sudden cardiac death. Common types of cardiomyopathy include hypertrophic cardiomyopathy, dilated cardiomyopathy, restrictive cardiomyopathy, arrhythmogenic right ventricular dysplasia, Takotsubo cardiomyopathy and diabetic cardiomyopathy. Each is associated with different aetiologies, symptoms and treatments.

Diabetic cardiomyopathy (DCM) is a multi-factorial specific heart that develops in around 50% of both type 1 and type 2 diabetes patients (Ritchie and Abel, 2020). Notably DCM is caused by specific diabetes-induced changes in the structure and function of the myocardium that are not directly attributable to other confounding factors such as coronary heart disease or hypertensions. Multiple mechanisms contribute to the pathogenesis of DCM including cell oxidative stress, moderate myocardial inflammation, mitochondrial dysfunction, apoptosis, and altered signalling pathways which leads to abnormalities in cardiomyocyte contractile properties, energy regulation and calcium homeostasis (Bugger and Abel, 2014; Salvatore et al., 2021). Among these are alterations in the expression levels of the regulatory and functional proteins involved in excitation-contraction coupling, such as SERCA2 and PLB. These factors progressively lead to ventricular dysfunction and heart failure, even in patients with efficient glycemic control. Despite the significant research attention devoted to the pathogenetic mechanisms underlying DCM, effective treatments which are able to prevent the initial changes occurring in the diabetic heart, before the appearance of overt signs of cardiac dysfunction, are still not available (Borghetti et al., 2018).

Green tea is a good source of catechins which have been found to exhibit powerful antioxidant, antiviral and anti-inflammatory properties (Ohishi et al, 2016). Green tea extracts are unique as they have few side effects, are inexpensive, and can be orally consumed. Polyphenolic compounds found in green tea include epigallocatechin-3-gallate (EGCG), epigallocatechin (EGC), epicatechin-3-gallate (ECG) and epicatechin (EC). Many of these compounds are able to restore the intracellular signalling pathways, which are altered at the initial stages of diabetes cardiomyopathy, leading to a recovery of cardiac functionality. Green tea catechins have been demonstrated to improve healthy cardiomyocyte mitochondrial function and energy availability and modulate the expression of key excitation-contraction coupling proteins (Bocchi et al. 2018).

Villela et al. 2020 reports that green tea extract increases ATP content and increases the SERCA2/PLB and p-PLB/PLB ratio in healthy rat cardiomyocytes. This paper concludes that these changes result in an improvement in cardiomyocyte mechanics. However, this paper does not demonstrate that catechins are effective in inducing these changes in experimental models of cardiomyopathy, or indeed diabetic cardiomyopathy which is associated with unique and specific factors. As such, this paper does not teach that catechins could be effective in treating or preventing diabetic cardiomyopathy.

Bocchi et al. 2018 reports that green tea extract increases ATP levels and increases the SERCA2/PLB and p-PLB/PLB ratio in healthy rat cardiomyocytes. As above, this paper does not consider that catechins could induce these changes in cardiomyopathic cells or diabetic cardiomyopathic cells and as such does not teach that catechins would be effective in treating or preventing diabetic cardiomyopathy.

Al Hroob et al. 2018 reviews pathophysiological mechanisms of diabetic cardiomyopathy and the known properties of EGCG. This paper concludes that EGCG might be a promising drug candidate for diabetic cardiomyopathy although it provides no specific data to support this conclusion which is thus entirely speculative. Further, this paper does not contemplate that catechins in general or a combination of catechins, such as Theaphenon® E (ThE) could have utility in treating or preventing diabetic cardiomyopathy.

There remains a need to identify novel therapies that reduce the risk of heart failure and sudden cardiac death in individuals with diabetes mellitus and/or diabetic cardiomyopathy. The present inventors have carried out a number of experiments and have discovered that catechins found in green tea are surprisingly effective at counteracting diabetes-induced changes to the myocardium and restoring normal cardiac performance and contractility in a rat model of early diabetes. They have also developed pharmaceutical formulations comprising a catechin component and have established an effective dosage regimen.

### SUMMARY OF THE INVENTION

The present inventors have discovered that treatment with the catechin containing composition ThE is able to reverse diabetes induced cardiomyocyte mitochondrial dysfunction and energy dysregulation and restore expression of key contractile proteins to normal in diabetic cardiomyocytes, hence restoring cardiac contractility. Furthermore, the present inventors have discovered that ThE treatment restores normal activity of citrate synthase and increases SIRT1 activity above control in diabetic cardiomyocytes. This is representative of restored mitochondrial function and protection against oxidative damage and inflammation.

Finally, the present inventors have shown that ThE treatment is able to restore normal cardiomyocyte mechanics and calcium transients in diabetic cardiomyocytes. These data suggest that formulations comprising a catechin component such as ThE is likely to be effective in treating or preventing diabetic cardiomyopathy.

Accordingly, the present invention provides a pharmaceutical formulation comprising a catechin component for use in the treatment or prevention of diabetic cardiomyopathy.

### BRIEF DESCRIPTION OF THE FIGURES

FIG. 1 shows the effect of THE treatment on the ATP content of cardiomyocytes in a STZ-induced diabetic rat model (Example 2).

FIGS. 2 (A-E) shows the effect of THE treatment on the level of expression of SERCA2 (2A), PLB (2B), and p-PLB (2C) and the effect of said treatment on the SERCA2/PLB (2D) and p-PLB/PLB (2E) ratio in early diabetic cardiomyocytes in a STZ-induced diabetic rat model (Example 3).

FIGS. 3 (A-B) shows the effect of THE treatment on the activity of citrate synthase (3A) and SIRT1 ratio in early diabetic cardiomyocytes in a STZ-induced diabetic rat model (Example 4).

FIGS. 4 (A-F) show the effect of THE treatment on cardiomyocyte mechanics and calcium transients in early diabetic cardiomyocytes in a STZ-induced diabetic rat model (Example 5).

### DETAILED DESCRIPTION OF THE INVENTION

In the present specification, the “dry weight” of a substance or component refers to the total mass of the substance or component excluding the mass of any solvent which may be included in the substance or component.

In the present specification, the terms “prophylaxis” and “prevention” are used interchangeably.

In a first aspect of the present invention there is provided a pharmaceutical formulation comprising a catechin component for use in the treatment or prevention of diabetic cardiomyopathy.

Catechins are typically found in tea extracts, particularly green tea extracts and therefore the catechin component may be a tea extract, particularly a green tea extract. Thus, the catechin component may be a catechin component comprising or consisting of catechins extracted or extractable from tea, particularly green tea. Other natural sources of catechins are possible e.g. from other plants such as pome fruits, cocoa or broad bean, the source typically being the fruit, pod, stem or leaf. The source of the catechin component is suitably a solid, dry component which does not comprise a solvent. The source of the catechin component may be synthetic.

The catechin component suitably comprises, consists essentially of or consists of epigallocatechin-3-gallate (EGCG), epigallocatechin (EGC), epicatechin-3-gallate (ECG) and epicatechin (EC) or a mixture thereof. In particular, the catechin suitably comprises, consists essentially of or consists of EGCG.

More suitably, the catechin component comprises, consists essentially of or consists of a mixture of at least two of the catechins listed above, for example the catechin component comprises, consists essentially of or consists of a mixture of EGCG and EGC. In particular, the catechin component comprises, consists essentially of or consists of a mixture of all of the above catechins, i.e. the catechin component comprises, consists essentially of or consists of a mixture of EGCG, EGC, ECG and EC.

The catechin component may optionally comprise gallocatechin gallate (GCG). The catechin component may optionally comprise (+/−) catechin (DL-C).

A particularly suitable catechin component for use in the present invention is sold under the trade mark Theaphenon® E, available from Tea Solutions, Hara Office, Inc, Tokyo, Japan.

The catechin component may comprise catechin(s) in an amount of 75% to 98% by weight, suitably 80% to 98% by weight, more suitably 85% to 95% by weight and typically about 90% by weight, with respect to the dry weight of the component.

The catechin component may, for example, comprises EGCG in an amount of 50% to 80% by weight, more suitably 55% to 75% by weight or 56% to 72% by weight, for example about 62% to 75% by weight, with respect to the dry weight of the component.

The catechin component may, for example, comprise EGC in an amount of 1% to 30% by weight, more suitably 2 to 25% by weight, for example 5% to 22% or 5% to 20% by weight, with respect to the dry weight of the component. In other cases, the amount of EGC may be from about 1% to 10% by weight, for example 2% to 9% by weight of the dry weight of the component.

The catechin component may, for example, comprise ECG in an amount of 1 to 10% by weight, for example 1 to 8% by weight or 1 to 6% by weight with respect to the dry weight of the component.

The catechin component may, for example, comprise EC in an amount of 1 to 15% by weight. In some suitable components, the amount of EC may be from about 1% to 5% by weight with respect to the dry weight of the component. In other suitable components, the amount of EC may be from about 7% to 15% by weight with respect to the dry weight of the component.

The catechin component may, for example, comprise GCG in an amount of up to 10% e.g. up to 8% e.g. 0.1-8% e.g. 0.1-5% e.g. 0.1-2% by weight with respect to the dry weight of the component.

The catechin component may, for example, comprise DL-C in an amount of up to 3% e.g. up to 2% e.g. 0.1-3% e.g. 0.1-2% e.g. 0.5-1.5% by weight with respect to the dry weight of the component.

The catechin component may, for example, comprise (i) EGCG in an amount of about 62% to 75% by weight (ii) EGC in an amount of 5% to 20% by weight (iii) ECG in an amount of 1% to 6% by weight and (iv) EC in an amount of 1% to 5% by weight, with respect to the dry weight of the component.

The catechin component may, for example, comprise (i) EGCG in an amount of about 62% to 75% by weight (ii) EGC in an amount of 5% to 20% by weight (iii) ECG in an amount of 1% to 6% by weight (iv) EC in an amount of 1% to 5% by weight (v) GCG in an amount of 0.1-8% and (vi) DL-C in an amount of 0.5% to 1.5% by weight, with respect to the dry weight of the component.

It is preferred that the catechin component should contain, at most, minimal amounts of non-catechin compounds which occur in tea extracts, for example compounds such as caffeine, theobromine and gallic acid. A typical catechin component for use according to the invention is preferably substantially free of caffeine, theobromine and gallic acid. For example, the catechin component may comprise less than 1% by weight (e.g. 0 to 1% by weight) caffeine and/or less than 1% by weight (e.g. 0 to 1% by weight) theobromine and/or less than 1% by weight (e.g. 0 to 1% by weight) gallic acid, where % by weight is expressed with respect to the dry weight of the component. Suitably, the amount of caffeine present in the catechin component is less than 0.5% by weight (e.g. 0 to 0.5% by weight), with respect to the dry weight of the component. In some cases, it is not possible to remove the caffeine, theobromine and/or gallic acid completely and so trace amounts may remain. Therefore, the catechin component may comprise 0.0001% to 1% by weight caffeine and/or 0.0001% to 1% by weight and/or 0.0001% to 1% by weight gallic acid, where the % by weight is with respect to the dry weight of the component.

The pharmaceutical formulations of the present invention, when formulated as a solid or discrete unit, may comprise a catechin component in an amount from 10% to 100% by weight, for example 20% to 100% by weight, for example 50% to 100% by weight, where the % by weight is with respect to the dry weight of the formulation. The pharmaceutical formulations of the present invention, when formulated in solution as a liquid, may comprise a catechin component in an amount from 0.01% to 50% by weight, for example 0.01% to 20% by weight, for example 0.1% to 10% by weight, for example 0.1% to 5% by weight, where the % by weight is with respect to the weight of the total formulation.

The pharmaceutical formulation of the invention may be formulated for administration by any route but suitably is adapted for oral administration; for administration by inhalation; or for administration to the oral or nasal mucosa.

The pharmaceutical formulation of the invention may be for use by oral administration; by administration by inhalation; or for administration to the oral or nasal mucosa.

Formulations for oral administration in the present invention may be presented as: discrete units such as capsules, sachets or tablets each containing a predetermined amount of the catechin component; as a powder or granules; as a solution or a suspension of the catechin component in an aqueous liquid or a non-aqueous liquid; or as an oil-in-water liquid emulsion or a water in oil liquid emulsion; or as a bolus etc. Said oral formulation may, in some cases be provided as a food, food additive or food supplement. Liquid formulations may, in some cases, be provided in the form of drinks, which may be provided in containers adapted to provide a single dose of the catechin component.

For formulations for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the catechin component in a free-flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the catechin component.

Other formulations suitable for oral administration include lozenges comprising the catechin component in a flavoured base, usually sucrose and acacia or tragacanth; pastilles comprising the catechin component in an inert base such as gelatin and glycerin, or sucrose and acacia; and mouthwashes comprising the catechin component in a suitable liquid carrier.

Particularly suitable oral formulations include discrete units such as capsules, tablets and sachets, especially tablets and capsules and more especially capsules.

Inhaled administration, i.e. topical administration to the lungs, may be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Alternatively, topical administration to the lung may be achieved by use of an aerosol formulation. Aerosol formulations typically comprise the catechin component suspended or dissolved in a suitable aerosol propellant, such as a chlorofluorocarbon (CFC), a hydrofluorocarbon (HFC) or a hydrofluoroolefin (HFO).

Particularly suitable inhaled formulations include formulations such as solutions adapted for inhalation via a nebuliser.

The mean daily dosage of the pharmaceutical formulations of the invention will depend upon various factors, such as the seriousness of the disease and the conditions of the patient (age, sex and weight). The skilled person may use technical means well known in the art in order to find the correct dosage amount and regime to ensure optimal treatment of each subject.

The pharmaceutical formulations of the invention may be administered to a patient once or more than once a day, for example two or three times a day. Such treatment may extend for a number of weeks or months.

The pharmaceutical formulations of the invention may be administered to a patient in an amount such that the dose of the catechin component according to the invention is from 10 mg to 3000 mg per day e.g. 10 mg to 2000 mg per day e.g. 10 mg to 1000 mg per day.

Orally administered pharmaceutical formulations of the invention may be administered to a patient in an amount such that the dose of the catechin component according to the invention is from 300 mg to 3000 mg per day e.g. from 600 mg to 2000 mg per day. In some cases, the oral dose of the catechin component may be from 600 mg to 1800 mg or from 600 mg to 1000 mg per day. Alternatively, the oral dose of the catechin component may be from 900 to 1800 mg/day e.g. 900 to 1500 mg/day. Alternatively, the oral dose of the catechin component may be from 100 mg to 2000 mg per day e.g. from 100 mg to 1000 mg per day e.g. from 300 mg to 1000 mg per day.

Pharmaceutical formulations of the invention which are administered by inhalation, especially inhalation via a nebuliser may be administered to a patient in an amount such that the dose of the catechin component may be from 10 mg to 60 mg per day, more suitably from 25 mg to 35 mg per day.

The present invention further provides a method for the treatment or prevention of diabetic cardiomyopathy comprising administering to a patient in need thereof a pharmaceutical formulation comprising a catechin component.

The present invention further provides a method for the treatment or prevention of diabetic cardiomyopathy comprising administering to a patient in need thereof a pharmaceutical formulation as defined above in an amount such that the dose of the catechin component may be from 10 mg to 3000 mg per day e.g. 10 mg to 2000 mg per day e.g. 10 mg to 1000 mg per day.

The invention further provides the use of a pharmaceutical formulation comprising a catechin component in the manufacture of a medicament for the treatment or prevention of diabetic cardiomyopathy.

The invention also provides the use of a pharmaceutical formulation as defined above in the manufacture of a medicament for the treatment or prevention of diabetic cardiomyopathy, wherein the pharmaceutical formulation is administered to a patient in an amount such that the dose of the catechin component may be from 10 mg to 3000 mg per day e.g. 10 mg to 2000 mg per day e.g. 10 mg to 1000 mg per day.

The pharmaceutical formulations for use according to the present invention are suitable for the treatment or prevention of diabetic cardiomyopathy.

The pharmaceutical formulations according to the present invention may be administered to a subject who has or is at risk of developing diabetes mellitus, for example the subject may have or be at risk of developing type 1 diabetes or type 2 diabetes. Suitably, the subject may have or be at risk of developing type 1 diabetes. In an alternative suitable aspect the subject may have or be at risk of developing type 2 diabetes.

The pharmaceutical formulations for use according to the present invention may be administered to a human subject.

### EXAMPLES

### Example 1-Development of a STZ-Induced Diabetic Rat Model

14-16-week old male Wistar rats (n=16), weighing 414±9.6 g (mean±standard error of the mean) were injected with a single intra-peritoneal (i.p.) injection of streptozocin (STZ, 60 mg/kg, Sigma-Aldrich, Milan, Italy). Control rats (n=9, CTRL) were not given such an injection. Glucose blood levels (mg/dl) and body weights (g) were measured in 4-h-fasting STZ-induced diabetic and control rats before and two days after the STZ injection, and then weekly until sacrifice. After a documented increase in blood glucose levels following STZ injection (two days after STZ injection), diabetic rats were either untreated (n=7, D) or subjected to daily Theaphenon® E (ThE; Tea Solutions, Hara Office, Inc, Tokyo, Japan) oral administration (n=9, D_GTE, 90 mg/day of ThE, a representative composition of which is set out in Table 3, dissolved in 50 ml of tap water) for 28 days. The effect of STZ injection and THE treatment is shown in Tables 1 and 2.

TABLE 1
Effect of STZ injection and THE treatment
on rat blood glucose (mg/dl)

| Day | Control | D | D_GTE |
|---|---|---|---|
| 0 | 103 ± 2 | 107 ± 6 | 105 ± 3 |
| 2 | 105 ± 4 | 356 ± 11 ** | 395 ± 28 ** |
| 14 | 101 ± 5 | 424 ± 25 ** | 448 ± 19 ** |
| 28 | 97 ± 5 | 472 ± 32 ** | 487 ± 3 ** |

** p < 0.01 vs control

TABLE 2
Effect of STZ injection and THE treatment on rat body weight (g)

| Day | Control | D | D_GTE |
|---|---|---|---|
| 0 | 359 ± 9 | 378 ± 5 | 384 ± 7 |
| 2 | 370 ± 9 | 355 ± 5 | 364 ± 8 |
| 14 | 382 ± 13 | 332 ± 6 ** | 335 ± 7 ** |
| 28 | 387 ± 14 | 331 ± 8 ** | 336 ± 9 ** |

** p < 0.01 vs control

TABLE 3
A Representative Composition of ThE

| Component | | % by weight |
|---|---|---|
| Total Catechins | | 94.87 |
| of which: | EGC (7.91%) | |
| | EC (2.63%) | |
| | DL-C (1.15%) | |
| | EGCG (73.92%) | |
| | GCG (7.34%) | |
| | ECG (1.92%) | |
| Caffeine | | 0.13 |
| Theobromine | | Trace |
| Gallic Acid | | Trace |

The results show that two days after STZ injection, the blood glucose levels were significantly increased in diabetic rats compared with control. During the subsequent weeks, blood glucose increased slightly in both untreated and ThE-treated diabetic rats but remained stable in control rats. Furthermore, a 10% decrease in body weight occurred in both treated and untreated diabetic rats during the first two weeks after STZ injection. Subsequently, the body mass exhibited only negligible changes while in control rats it slightly increased.

### Example 2-Effect of ThE on ATP Content of Early Diabetic Cardiomyocytes in a STZ-Induced Diabetic Rat Model

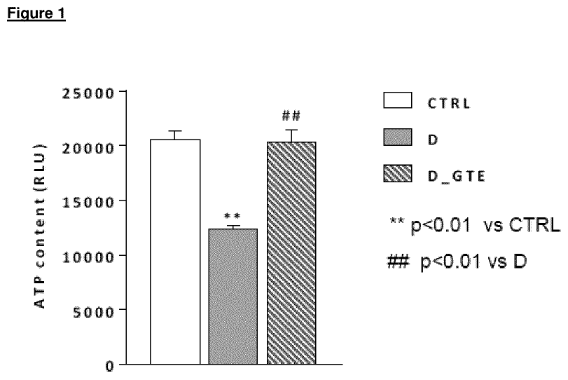

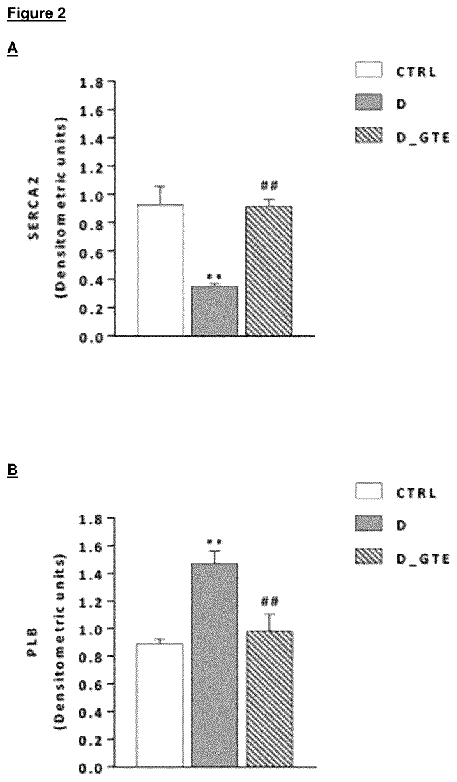

Control, STZ-induced diabetic ThE untreated (D) and STZ-induced diabetic ThE treated (D_GTE) rats (treatment per Example 1) were anaesthetised with ketamine chloride (Imalgene, Merial, Milan, Italy; 40 mg/kg i.p.) and medetomidine hydrochloride (Domitor, Pfizer Italia S.r.l., Latina, Italy; 0.15 mg/kg i.p.), and sacrificed. The heart was rapidly excised and left ventricular cardiomyocytes were enzymatically isolated by collagenase perfusion. The heart was mounted on a Langendorff apparatus and retrogradely perfused, at 37° C., through the aorta with the following sequence of solutions gassed with 100% oxygen: (1) a calcium-free solution (126 NaCl mM, 22 Mm dextrose, 5.0 mM MgCl₂, 4.4 mM KCl, 20 mM taurine, 5 mM creatine, 5 mM sodium pyruvate, 1 mM NaH₂PO₄, and 24 mM HEPES; pH=7.4, adjusted with NaOH) for 5 min to remove the blood, (2) a low-calcium solution (0.1 mM) plus 1 mg/ml type 2 collagenase (Worthington Biochemical Corporation, Lakewood, NJ, USA) and 0.1 mg/ml type XIV protease (Sigma-Aldrich) for about 15 min, and (3) an enzyme free, low-calcium solution for 5 min. The left ventricle was then minced, shaken for 10 min and the cells were filtered through a nylon mesh. Cells were washed three times with low-calcium solution (0.1 mM) and centrifuged (42 g for 5 min). The supernatant was removed and the pellet was stored at −80° C. The ATP intracellular content was measured by the Cell Titer Glo® Luminescent Cell Viability (Promega, Milan, Italy) according to the manufacturer's protocol. Luminescence intensity was measured by the EnSpire® multimode plate reader (PerkinElmer, Waltham, MA, USA). The raw luminescence data, given in relative light units (RLUs), was normalized to the total protein content of each sample, measured by the DC Protein assay kit (Bio-Rad, Hercules, CA, USA). The results are shown in FIG. 1.

The results in FIG. 1 demonstrate that ATP levels in STZ-induced diabetic rat cardiomyocytes are greatly reduced compared to control rats as a consequence of diabetes-induced impairment of cardiomyocyte mitochondrial function and energy regulation. However, ATP levels in ventricular cardiomyocytes were restored to normal in STZ-induced diabetic rats subjected to daily ThE administration.

### Example 3-Effect of ThE on Expression of Key Contractile Proteins in Diabetic Cardiomyocytes in a STZ-Induced Diabetic Rat Model

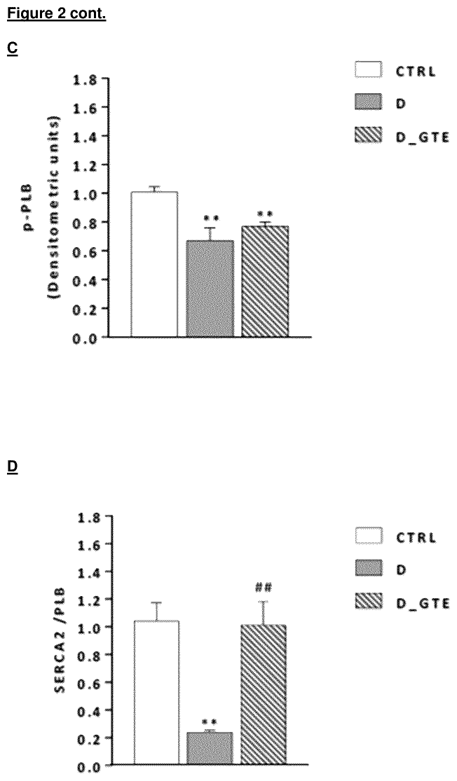

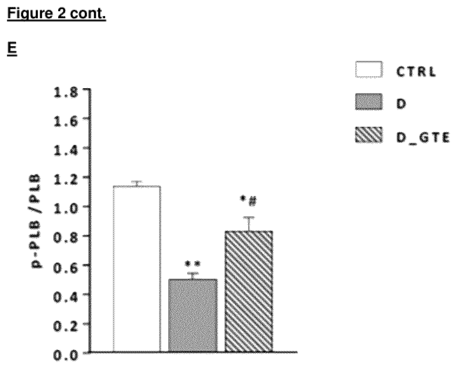

Control, STZ-induced diabetic ThE untreated (D) and STZ-induced diabetic ThE treated (D_GTE) rat cardiomyocytes (preparation per Example 2) were finely ground into liquid nitrogen. 30 mg of powder was homogenised in 500 μL of ice-cold Radioimmunoprecipitation Assay Buffer (RIPA) supplemented with protease and phosphatase inhibitor cocktails (Sigma-Aldrich, Milan, Italy). In addition, ice-cold RIPA buffer plus inhibitors was used to lyse isolated cardiomyocytes. 30 μg of protein lysate was separated by SDS-PAGE, blotted onto PVDF or nitrocellulose membranes, and quantified by immunodetection analysis. After blocking in a solution of 2% bovine serum albumin (BSA), the membranes were incubated with primary antibodies: rabbit polyclonal anti-phospho-phospholamban (p-PLB) (EMD Millipore Corporation, Temecula, CA, code 07-052), dilution 1:200; mouse monoclonal anti-phospholamban (PLB) (Abcam, Cambridge, UK, code ab2865), dilution 1:1000; rabbit polyclonal anti-SERCA2 ATPase (Abcam, Cambridge, UK, code ab3625), dilution 1:1000; mouse monoclonal anti-actin (Santa-Cruz Biotechnology, Santa Cruz, CA, USA, code sc-81178) dilution 1:500. Detection of the immunoreactive bands was achieved using horseradish peroxidase-conjugated anti-mouse (dilution 1:5000) or anti-rabbit (dilution 1:200,000) secondary antibodies (Sigma-Aldrich, Milan, Italy, catalogue numbers are A5906 and A0545, respectively) and the BM Chemiluminescence Blotting Substrate (Hoffmann-La Roche, Basel, Switzerland Catalogue number 11 500 694 00). The expression levels of SERCA2, PLB, and p-PLB were measured by densitometric analysis and normalized to B-actin. Densitometry was performed by the Quantity One analysis software (Bio-Rad). The result are shown in FIGS. 2 (A-E).

The results in FIGS. 2 (A-E) demonstrate that in STZ-induced diabetic rat cardiomyocytes expression of SERCA2, and to a lesser extent p-PLB, is significantly reduced in comparison to control, whilst the expression of PLB is significantly increased in comparison to control. This has the effect of causing a significant reduction in both the SERCA2/PLB and p-PLB/PLB ratios. Note that the SERCA2/PLB ratio is reported to be critical for defining the affinity of SERCA2 for calcium yielding positive effects on cardiomyocyte contractility, while a decreased ratio has the opposite effect (Koss et al. 1997). Note that phosphorylation of PLB to produce p-PLB relieves PLB mediated inhibition of SERCA2 activity and an increased p-PLB/PLB ratio is associated with improved cardiac function (MacLennan et al. 2003). As such, STZ-induced diabetic rats have dysregulated expression of key contractile proteins and consequently impaired cardiac function.

The results in FIGS. 2 (A-E) also demonstrate that STZ-induced diabetic ThE treated rat cardiomyocytes express SERCA2 and PLB at comparable levels to control. As such, the SERCA2/PLB and p-PLB/PLB ratios in STZ-induced diabetic ThE treated rat cardiomyocytes are normal. Thus, ThE treatment reverses diabetes induced dysfunction of cardiomyocytes.

### Example 4-Effect of ThE on the Activity of Citrate Synthase and SIRT1 in a STZ-Induced Diabetic Rat Model

Following sacrifice of control, STZ-induced diabetic ThE untreated and STZ-induced diabetic ThE treated rats (treatment per Example 1), the heart was excised and perfused with a 0.9% NaCl solution at 37° C. to drain the residual blood. Then, the tissues (left and right ventricles) were snap frozen in liquid nitrogen and stored at −80° C. These tissues were ground into liquid nitrogen and used to determine the activity of the citrate synthase and SIRT1 enzymes.

Citrate synthase is a key mitochondrial enzyme as it catalyses the condensation of oxaloacetate and acetyl-CoA to citrate in the mitochondrial matrix, a reaction which is often seen as the initiating reaction in the citric acid cycle. Thus, the activity of this enzyme can serve as measure of mitochondrial function in a cell. SIRT1, together with AMPK, is responsible for the regulation of the transcriptional co-activator peroxisome proliferator-activated receptor-gamma co-activator-1α (PGC1α), which is the main regulator of mitochondrial biogenesis and function. SIRT1 is also responsible for modulating the activity of several transcription factors which are responsible for the activation of antioxidant response elements and inhibition of the NF-κB-mediated pro-inflammatory pathway (Ruderman et al. 2010). SIRT1 is found at decreased levels in diabetic cardiomyocytes and is thus associated with allowing oxidative damage and inflammation.

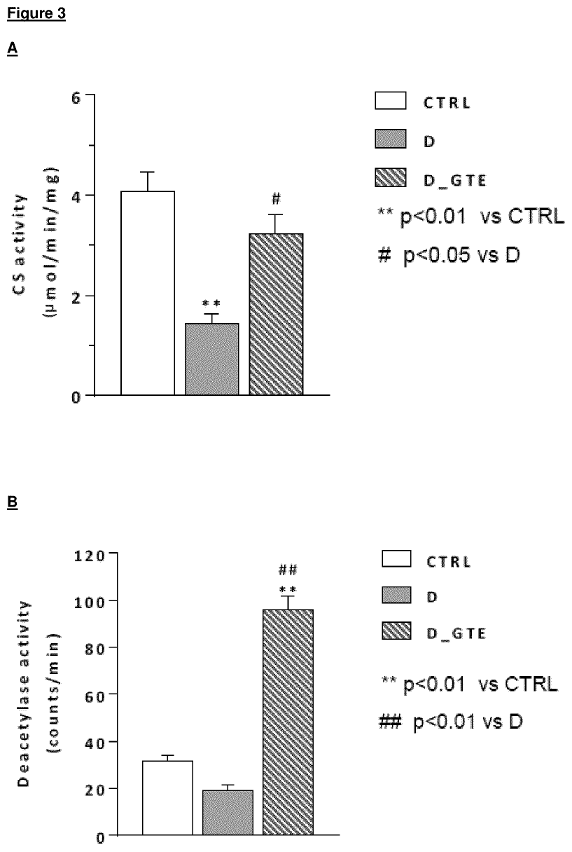

Citrate synthase activity in control, STZ-induced diabetic ThE untreated (D) and STZ-induced diabetic ThE treated (D_GTE) rat cardiomyocytes was detected by using MitoCheck® Citrate Synthase Activity Assay Kit (Cayman Chemical, Michigan, USA) according to the manufacturer's protocol. This assay measures the production of SH-CoA by monitoring the absorbance of Citrate Synthase Developing Reagent at 412 nm. Absorbance intensity was measured at 30 sec intervals for 20 min at 25° C. using the EnSpire® multimode plate reader (PerkinElmer). SIRT1 activity in control, STZ-induced diabetic ThE untreated (D) and STZ-induced diabetic ThE treated (D_GTE) rat cardiomyocytes was measured using a SIRT1 fluorometric assay kit (Abcam, Cambridge, UK) according to the manufacturer's instructions. The fluorescence intensity (Ex355 nm/Em460 nm) was measured at 2 min intervals for 60 min using the EnSpire® multimode plate reader (PerkinElmer). The results are shown in FIGS. 3A and 3B respectively.

The results in FIG. 3A demonstrate that citrate synthase activity in STZ-induced diabetic rat cardiomyocytes is significantly reduced in comparison to control cardiomyocytes. However, citrate synthase activity in STZ-induced diabetic ThE treated rat cardiomyocytes is comparable to control cardiomyocytes i.e. THE treatment restores citrate synthase activity and thus restores mitochondrial function.

Similarly, the results in FIG. 3B demonstrate that SIRT1 activity in STZ-induced diabetic rat cardiomyocytes is reduced in comparison to control cardiomyocytes. However, SIRT1 activity in STZ-induced diabetic ThE treated rat cardiomyocytes is significantly increased in comparison to control cardiomyocytes i.e. THE treatment not only restores, but improves SIRT1 activity compared to control, thus preventing oxidative damage and pro-inflammatory signalling.

### Example 5-Effect of ThE on Cardiomyocyte Mechanics and Calcium Homeostasis in a STZ-Induced Diabetic Rat Model

Following sacrifice of control, STZ-induced diabetic ThE untreated and STZ-induced diabetic ThE treated rats (treatment per Example 1), the heart was rapidly excised and left ventricular cardiomyocytes were enzymatically isolated by collagenase perfusion. The left ventricle was then minced and shaken for 10 min. The cells were filtered through a nylon mesh and a fraction were re-suspended in a low-calcium solution for 20 min, before gradually being brought to 1 mM Ca²⁺ in about 80 min, and then used for measuring sarcomere shortening and calcium transients.

Mechanical properties and calcium transients were evaluated by using the IonOptix fluorescence and contractility systems (IonOptix, Milton, MA, USA). The isolated left ventricular cardiomyocytes were placed in a chamber mounted on the stage of an inverted microscope (Nikon-Eclipse TE2000-U, Nikon Instruments, Florence, Italy) and perfused at 1 mL/min at 37° C. with a Tyrode solution (140 mM NaCl, 5.4 mM KCl, 1 mM MgCl₂, 5 mM HEPES, 5.5 mM glucose, and 1 mM CaCl₂), pH 7.4, adjusted with NaOH; all chemicals from Sigma-Aldrich). Rod-shaped myocytes with clear edges and an average sarcomere length ≥1.7 μm were selected for the analysis, using a 40× oil objective lens. None of the selected cardiomyocytes showed spontaneous contractions. Cells were field-stimulated at a frequency of 0.5 Hz by constant current pulses (2 ms in duration and twice diastolic threshold in intensity; MyoPacer Field Stimulator, IonOptix), delivered by platinum electrodes placed on opposite sides of the chamber, connected to a MyoPacer Field Stimulator (IonOptix). Load-free contractions of myocytes were measured with the IonOptix system, which captures sarcomere length dynamics via a Fast Fourier Transform algorithm.

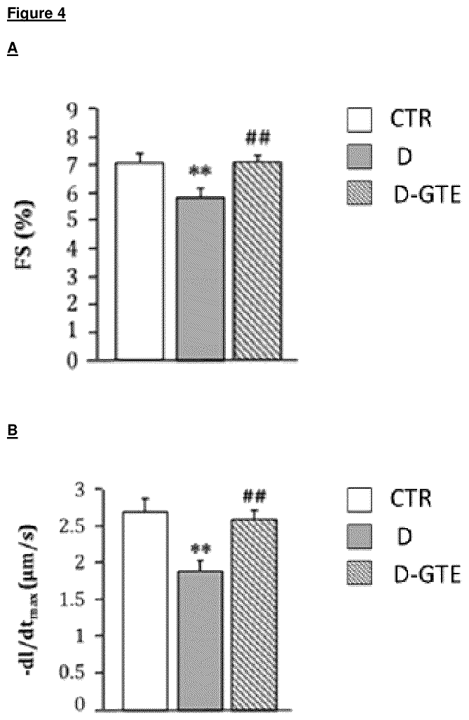

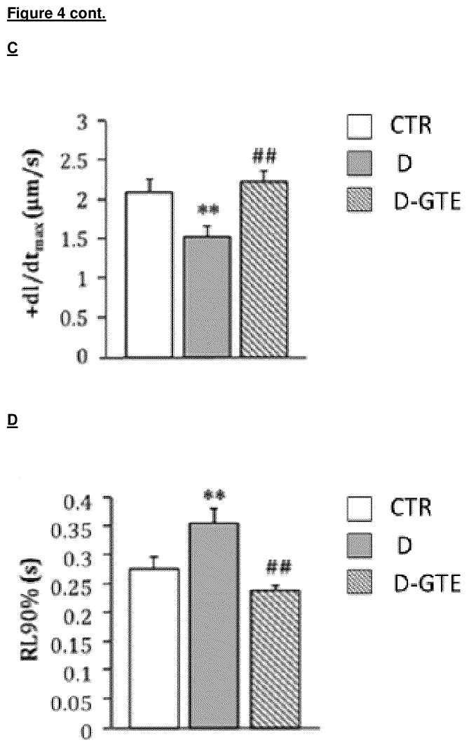

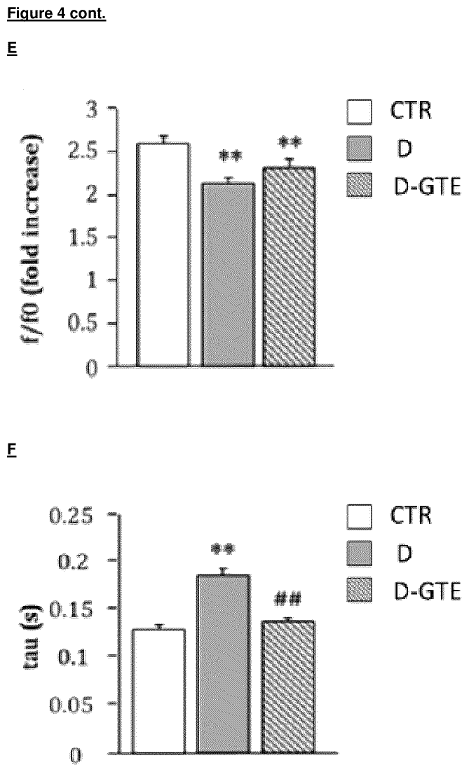

Cell contractile properties and calcium dynamics were simultaneously recorded by loading the cardiomyocytes with Fluo-3 AM (5 μM; Thermo Fisher Scientific, Waltham, MA, USA), previously mixed with Pluronic™ F-127 (10% final concentration; Thermo Fisher Scientific, Waltham, MA, USA), for 20 min. The results are shown in FIGS. 4 (A-F).

The results in FIGS. 4 (A-F) demonstrate that ThE treatment facilitates a complete recovery of cardiomyocyte mechanics in a STZ-induced diabetic rat model. For example, THE treatment supported recovery of the fraction of shortening (FS, FIG. 4A), the maximal rate of shortening (−dl/dtₘₐₓ, FIG. 4B) and re-lengthening (+dl/dtₘₐₓ, FIG. 4C), resulting in shorter relaxation times as measured at 90% of re-lengthening (RL, FIG. 4D). Furthermore, ThE treatment restores normal calcium signalling in a STZ-induced diabetic rat model, as represented by recovery of the calcium transient amplitude expressed as peak fluorescence normalized to baseline fluorescence (f/f0, FIG. 4E), and time constant of the intracellular calcium decay (tau, FIG. 4F).

### Summary of Biological Data

Rats can be injected with streptozocin to produce a representative model of diabetes (Example 1).

ThE treatment is able to reverse diabetes induced cardiomyocyte mitochondrial dysfunction and energy dysregulation (Example 2).

ThE treatment is able to restore expression of key contractile proteins to normal in diabetic cardiomyocytes, hence restoring cardiac contractility (Example 3).

ThE treatment restores normal activity of citrate synthase and increases SIRT1 activity above control in diabetic cardiomyocytes. This is representative of restored mitochondrial function and protection against oxidative damage and inflammation (Example 4).

ThE treatment is able to restore normal cardiomyocyte mechanics and calcium transients in diabetic cardiomyocytes (Example 5).

Collectively the data suggest that formulations comprising a catechin component such as ThE is likely to be effective in treating or preventing diabetic cardiomyopathy.

### REFERENCES

Al Hroob A M, Abukhalil M H, Hussein O E, Mahmoud A M. Pathophysiological mechanisms of diabetic cardiomyopathy and the therapeutic potential of epigallocatechin-3-gallate. Biomed Pharmacother. 2019; 109:2155-2172.
Bocchi L, Savi M, Naponelli V, et al. Long-Term Oral Administration of Theaphenon-E Improves Cardiomyocyte Mechanics and Calcium Dynamics by Affecting Phospholamban Phosphorylation and ATP Production. Cell Physiol Biochem. 2018; 47 (3): 1230-1243.
Borghetti G, von Lewinski D, Eaton D M, Sourij H, Houser S R, Wallner M. Diabetic Cardiomyopathy: Current and Future Therapies. Beyond Glycemic Control. Front Physiol. 2018. 9:1514.
Bugger H, Abel E D. Molecular mechanisms of diabetic cardiomyopathy. Diabetologia. 2014. 57 (4): 660-671.
Koss, K. L., Grupp, I. L. & Kranias, E. G. The relative phospholamban and SERCA2 ratio: a critical determinant of myocardial contractility. Basic Res Cardiol. 1991. 92, 17-24.
MacLennan D H, Kranias E G. Phospholamban: a crucial regulator of cardiac contractility. Nat Rev Mol Cell Biol. 2003. 4 (7): 566-577.
Ohishi, T.; Goto, S.; Monira, P.; Isemura, M.; Nakamura, Y. Anti-inflammatory Action of Green Tea. Antiinflamm. Antiallergy. Agents Med. Chem. 2016, 15, 74-90.
Ritchie R H, Abel E D. Basic Mechanisms of Diabetic Heart Disease. Circ Res. 2020.126 (11): 1501-1525.
Ruderman N B, Xu X J, Nelson L, et al. AMPK and SIRT1: a long-standing partnership?. Am J Physiol Endocrinol Metab. 2010. 298 (4): E751-E760.
Salvatore T, Pafundi P C, Galiero R, et al. The Diabetic Cardiomyopathy: The Contributing Pathophysiological Mechanisms. Front Med (Lausanne). 2021. 8:695792.
Vilella R, Sgarbi G, Naponelli V, et al. Effects of Standardized Green Tea Extract and Its Main Component, EGCG, on Mitochondrial Function and Contractile Performance of Healthy Rat Cardiomyocytes. Nutrients. 2020; 12 (10): 2949.

1-17. (canceled)

18. A method for the treatment or prevention of diabetic cardiomyopathy comprising administering to a patient in need thereof a pharmaceutical formulation comprising a catechin component.

19. (canceled)

20. The method according to claim 18, wherein the catechin component comprises, consists essentially of or consists of epigallocatechin-3-gallate (EGCG), epigallocatechin (EGC), epicatechin-3-gallate (ECG) and epicatechin (EC) or a mixture thereof.

21. The method according to claim 20, wherein the catechin component comprises, consists essentially of or consists of EGCG.

22. The method according to claim 18, wherein the catechin component comprises catechin in an amount of 75 to 98% by weight with respect to the dry weight of the component.

23. The method according to claim 20, wherein the catechin component comprises EGCG in an amount of 50% to 80% by weight with respect to the dry weight of the component.

24. The method according to claim 20, wherein the catechin component comprises EGC in an amount of 1% to 30% by weight with respect to the dry weight of the component.

25. The method according to claim 20, wherein the catechin component comprises ECG in an amount of 1% to 10% by weight with respect to the dry weight of the component.

26. The method according to claim 20, wherein the catechin component comprises EC in an amount of 1% to 15% by weight with respect to the dry weight of the component.

27. The method according to claim 18, wherein the catechin component comprises GCG in an amount of up to 10% by weight with respect to the dry weight of the component.

28. The method according to claim 18, wherein the catechin component comprises DL-C in an amount of up to 3% by weight with respect to the dry weight of the component.

29. The method according to claim 18, wherein the catechin component comprises 0 to 1% caffeine, 0 to 1% theobromine and 0 to 1% gallic acid by weight with respect to the dry weight of the component.

30. The method according to claim 18, wherein the catechin component comprises 0 to 0.5% caffeine by weight with respect to the dry weight of the component.

31. The method according to claim 18, wherein the formulation is formulated as a solid or discrete unit and comprises a catechin component in an amount from 10% to 100% by weight, where the % by weight is with respect to the dry weight of the formulation.

32. The method according to claim 18, wherein the formulation is formulated in solution as a liquid and comprises a catechin component in an amount from 0.01% to 50% by weight, where the % by weight is with respect to the weight of the total formulation.

33. The method according to claim 18, wherein the administering is selected from orally administering; administering by inhalation; and administering to the oral or nasal mucosa.

34. The method according to claim 33, wherein the administering is orally administering and wherein the pharmaceutical formulation is adapted for oral administration.

35. The method according to claim 33, wherein the administering is orally administering to a patient in an amount such that the dose of the catechin component is from 300 mg to 3000 mg per day.
